(19) United States
(12) Patent Application Publication
(10) Pub. No.: US 2011/0136868 A1
(43) Pub. Date: Jun. 9, 2011

(54) AGENTS FOR INHIBITING OSTEOCLASTOGENESIS AND/OR OSTEOCLAST ACTIVATION

(75) Inventors: Anastasios Karadimitris, London (GB); Nikki Horwood, South Woodford (GB); Amin Rahemtulla, Summertown (GB); Anne Dell, Middlesex (GB); Terry Butters, Garsington (GB); Raymond Dwek, Oxford (GB)

(73) Assignee: University of Oxford

(21) Appl. No.: 12/847,038

(22) Filed: Jul. 30, 2010

Related U.S. Application Data
(60) Provisional application No. 61/282,033, filed on Dec. 7, 2009.

(51) Int. Cl. A61K 31/445; A61P 35/00; A61P 19/00

(52) U.S. Cl. 514/319; 514/328; 514/315

(57) ABSTRACT

Agents, which are both ceramide glucosyltransferase inhibitors and glucosidase inhibitors, may inhibit osteoclastogenesis and/or reduce osteoclast activation and, therefore, may be useful for osteolytic activity and bone loss in subjects with conditions, such as multiple myeloma.

### CROSS-REFERENCE TO RELATED APPLICATIONS

This application claims priority from U.S. Provisional Patent Application No. 61/282,033, filed Dec. 7, 2009. The entire contents of the aforementioned application are incorporated herein by reference.

### FIELD

The present disclosure relates in general to the use of iminosugars for medical purposes and, in particular, to the use of iminosugars for inhibiting osteoclastogenesis and/or osteoclast activation.

### SUMMARY

According to one embodiment, a method for inhibiting osteoclastogenesis and/or reducing osteoclast activation, comprises administering to a subject in need thereof an effective amount of an agent, which is a ceramide glucosyltransferase inhibitor and a glucosidase inhibitor.

According to another embodiment, a method of reducing or preventing osteolytic activity and/or bone loss, comprises administering to a subject in need thereof an effective amount of an agent, which is a ceramide glucosyltransferase inhibitor and a glucosidase inhibitor.

### DRAWINGS

The application file contains at least one drawing executed in color. Copies of this patent application publication with color drawing(s) will be provided by the Office upon request and payment of the necessary fee.

FIG. 1 A-B present data for in vitro inhibition by selected iminosugars of RANKL-dependent osteoclastogenesis.

FIG. 2 A-D present data for inhibition of MAPK signaling and NFATc activation during osteoclastogenesis for selected iminosugars.

FIG. 3 presents data related to glycosphyngolipids perturbation of association of Src and TRAF6 with rafts.

FIG. 4 A-B presents data for in vivo inhibition by selected iminosugars of osteoclast activation by galactosylceramide and RANKL.

FIG. 5 A-B present mass spectral profiles of GSL in multiple myeloma (MM) patients. The profiles reveal that GM2 and GM3 are most prevalent GSL in MM.

FIG. 6 A-E show data demonstrating that GM3 cooperates with RANKL and IGF-1 in promoting osteoclastogenesis.

### DETAILED DESCRIPTION

Unless otherwise specified “a” or “an” means one or more.

Osteoclast is the primary bone-resorbing cell in both normal and pathologic states. Increased osteoclastic bone resorption can result from both increased osteoclast formation and activation of preformed osteoclasts to resorb bone. In patients with bone metastases, osteolytic bone destruction can result in severe bone pain, pathologic fractures, hypercalcemia, and nerve compression syndromes. Several tumors show a high predilection for bone, including renal cancer, lung cancer, thyroid cancer, prostate cancer, multiple myeloma and breast cancer, see e.g. Roodman, Journal of Clinical Oncology, vol. 19, 2001, p. 3562. Osteoclast formation and activation may also contribute to osteolytic disease and bone loss in individuals suffering from osteoporosis, such as post-menopausal osteoporosis, Paget's disease, rheumatoid arthritis and head and neck squamous cell carcinoma, see e.g. U.S. Pat. No. 7,462,646.

Inhibition of glycosphingolipids by D-threo-1-phenyl-2-decanoylamin-3-morpholino-1-propanol (d-PDMP), which is a glucosylceramide synthase inhibitor, can inhibit the osteoclast formation induced by macrophage-colony stimulating factor and receptor activator of nuclear factor-κB ligand (RANKL), see Iwamoto et al., Journal of Biological Chemistry, 276, 46031-46038, 2001.

The present inventors discovered that inhibition of glycosphingolipids per se by a particular agent may be not sufficient by itself for this agent to be effective in inhibiting osteoclastogenesis and reducing osteoclast activation. Thus, to be effective in inhibiting osteoclastogenesis and/or reducing osteoclast activation, in addition to being a ceramide glucosyltransferase (CGT) inhibitor, an agent should also be an inhibitor of one or more additional enzymes, which are other than CGT.

In many embodiments, an agent, which can be effective in inhibiting osteoclastogenesis and/or reducing osteoclast activation, may be both a CGT inhibitor and a glucosidase inhibitor, i.e. the agent can an inhibitory effect on both CGT and glucosidase. The term “glucosidase inhibitor” means an agent, which can have an inhibitory activity on at least one of α-glucosidase and β-glucosidase. In many embodiments, the agent, that is both a CGT inhibitor and a glucosidase inhibitor, may be an iminosugar, such as N-substituted deoxynojirimycin.

In certain embodiments, the agent, that is both a CGT inhibitor and a glucosidase inhibitor, may be a compound of formula I, or a pharmaceutically acceptable salt or a prodrug of such compound:

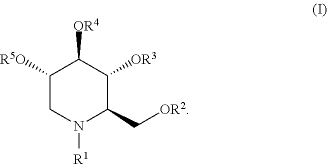

In formula (I), R¹ may be selected from alkyls, cycloalkyls, aryls, alkenyls, acyls, aralkyls, aroyls, alkoxy groups, aralkoxy groups and heterocyclic groups; while R², R³, R⁴, and R⁵ may be each independently selected from hydrogen, acyl groups, alkanoyl groups, aroyl groups, and haloalkanoyl groups.

In some embodiments, R¹ may be substituted or unsubstituted, branched or unbranched alkyl groups comprise from 1 to 24 carbon atoms, or from 2 to 12 carbon atoms or from 3 to 5 carbon atoms or from 14 to 22 carbon atoms or from 17 to 20 carbon atoms.

As used herein, the term “alkyl”, alone or in combination, means a straight-chain or branched-chain alkyl radical containing from 1 to and including 24 carbon atoms. Substituted alkyl, alone or in combination, means an alkyl radical which is optionally substituted as defined herein with respect to the definitions of aryl and heterocyclo. Alkylene means a saturated aliphatic hydrocarbon moiety attached at two or more positions, such as methylene (—CH₂—). Examples of alkyl radicals include methyl, ethyl, n-propyl, isopropyl, n-butyl, isobutyl, sec-butyl, tert-butyl, pentyl, iso-amyl, hexyl, octyl, nonyl, decyl, unadecyl, octadecyl and the like.

The term “cycloalkyl”, alone or in combination, means a saturated or partially saturated monocyclic, bicyclic or tricyclic alkyl radical wherein each cyclic moiety contains preferably from 3 to 10 carbon atom ring members and which may optionally be a benzo fused ring system which is optionally substituted as defined herein with respect to the definition of aryl. Examples of such cycloalkyl radicals include cyclopropyl, cyclobutyl, cyclopentyl, cyclohexyl, cycloheptyl, octahydronaphthyl, 2,3-dihydro-1H-indenyl, adamantyl and the like.

The term “aryl”, alone or in combination, or “ara” or” “ar” in combination, means a phenyl or naphthyl radical which is optionally substituted with one or more substituents selected from the group consisting of alkyl, alkylcarbonyl, alkoxy, halogen, hydroxy, amino, nitro, cyano, haloalkyl, haloalkylthio, haloalkyloxy, carboxy, alkoxycarbonyl, cycloalkyl, heterocyclo, alkylcarbonylamino, aminoalkanoyl, amido, aminocarbonyl, arylcarbonyl, arylcarbonylamino, aryl, aryloxy, alkyloxycarbonyl, arylalkyloxycarbonyl, alkoxycarbonylamino, substituted amino, disubstituted amino, substituted aminocarbonyl, disubstituted aminocarbonyl, substituted amido, disubstitutedamido, aralkoxycarbonylamino, alkylthio, alkylsulfinyl, alkylsulfonyl, haloalkylthio, haloalkylsulfinyl, haloalkylsulfonyl, arylthio, arylsulfinyl, arylsulfonyl, alkylsulfinylamino, alkylsulfonylamino, haloalkylsulfinylamino, haloalkylsulfonylamino, arylsulfinylamino, arylsulfonylamino, heterocyclo, sulfonate, sulfonic acid, trisubstitutedsilyl and the like. It is intended to include both fused ring systems, such as naphthyl and .beta.-carbolinyl, and substituted ring systems, such as biphenyl, phenylpyridyl, naphthyl and diphenylpiperazinyl. Examples of aryl radicals are phenyl, p-tolyl, 4-methoxyphenyl, 4-(tert-butoxy)phenyl, 3-methyl-4-methoxyphenyl, 4-fluorophenyl, 4-chlorophenyl, 3-nitrophenyl, 3-aminophenyl, 3-acetamidophenyl, 4-acetamidophenyl, 2-methyl-3-acetamidophenyl, 4-CF₃-phenyl, 2-methyl-3-aminophenyl, 4-CF₃O-phenyl, 3-methyl-4-aminophenyl, 2-amino-3-methylphenyl, 2,4-dimethyl-3-aminophenyl, 4-hydroxyphenyl, 3-methyl-4-hydroxyphenyl, 1-naphthyl, 2-naphthyl, 3-amino-1-naphthyl, 2-methyl-3-amino-1-naphthyl, 6-amino-2-naphthyl, 4,6-dimethoxy-2-naphthyl, piperazinylphenyl and the like.

The terms “aralkyl” and “aralkoxy”, alone or in combination, means an alkyl or alkoxy radical as defined above in which at least one hydrogen atom is replaced by an aryl radical as defined above. Thus, “aryl” includes substituents such as benzyl, 2-phenylethyl, dibenzylmethyl, hydroxyphenylmethyl, methylphenylmethyl, and diphenylmethyl, and “aryloxy” includes substituents such as benzyloxy, diphenylmethoxy, 4-methoxyphenylmethoxy and the like.

The term “aroyl” means an acyl radical derived from an arylcarboxylic acid, “aryl” having the meaning given above. Examples of such aroyl radicals include substituted and unsubstituted benzoyl or napthoyl such as benzoyl, 4-chlorobenzoyl, 4-carboxybenzoyl, 4-(benzyloxycarbonyl)benzoyl, 1-naphthoyl, 2-naphthoyl, 6-carboxy-2-naphthoyl, 6-(benzyloxycarbonyl)-2-naphthoyl, 3-benzyloxy-2-naphthoyl, 3-hydroxy-2-naphthoyl, 3-(benzyloxyformamido)-2-naphthoyl, and the like.

Methods of synthesizing compounds of formula (I) are known and are described, for example, in U.S. Pat. Nos. 5,622,972, 4,246,345, 4,266,025, 4,405,714, and 4,806,650.

In some embodiments, the agent, that is both a CGT inhibitor and a glucosidase inhibitor, can be in a form of a salt derived from an inorganic or organic acid. Pharmaceutically acceptable salts and methods for preparing salt forms are disclosed, for example, in Berge et al. (J. Pharm. Sci. 66:1-18, 1977). Examples of appropriate salts include but are not limited to the following salts: acetate, adipate, alginate, citrate, aspartate, benzoate, benzenesulfonate, bisulfate, butyrate, camphorate, camphorsulfonate, digluconate, cyclopentanepropionate, dodecylsulfate, ethanesulfonate, glucoheptanoate, glycerophosphate, hemisulfate, heptanoate, hexanoate, fumarate, hydrochloride, hydrobromide, hydroiodide, 2-hydroxyethanesulfonate, lactate, maleate, methanesulfonate, nicotinate, 2-naphthalenesulfonate, oxalate, palmoate, pectinate, persulfate, 3-phenylpropionate, picrate, pivalate, propionate, succinate, tartrate, thiocyanate, tosylate, mesylate, and undecanoate.

In some embodiments, the agent, that is both a CGT inhibitor and a glucosidase inhibitor, may also used in a form of a prodrug. Prodrugs of DNJ derivatives, such as the 6-phosphorylated DNJ derivatives, are disclosed in U.S. Pat. Nos. 5,043,273 and 5,103,008. In some embodiments, the agent, that is both a CGT inhibitor and a glucosidase inhibitor, may be used as a part of a composition, which further comprises a pharmaceutically acceptable carrier and/or a component useful for delivering the composition to an animal. Numerous pharmaceutically acceptable carriers useful for delivering the compositions to a human and components useful for delivering the composition to other animals, such as cattle are known in the art. Addition of such carriers and components is well within the level of ordinary skill in the art.

In some embodiments, the iminosugar, such as the compound of formula (I), may be used in a liposome composition, such as those disclosed in US publication 2008/0138351; U.S. application Ser. No. 12/410,750 filed Mar. 25, 2009 and U.S. provisional application No. 61/202,699 filed Mar. 27, 2009.

In some embodiments, the agent, which is both a CGT inhibitor and a glucosidase inhibitor, may be administered to a cell culture in order to inhibit osteoclastogenesis and/or reduce osteoclast activation in the cells. In some embodiments, the agent may be administered to an animal, such as a human being, in order to treat or prevent a condition, which may be progressing via osteoclastogenesis and/or osteoclast activation. Examples of such conditions include osteolytic disease and/or bone loss or destruction in subjects with renal cancer, lung cancer, thyroid cancer, prostate cancer, multiple myeloma, breast cancer, osteoporosis, such as post-menopausal osteoporosis, Paget's disease, rheumatoid arthritis or head and neck squamous cell carcinoma.

The amount of the agent administered to a cell, or an animal can be an amount effective to inhibit osteoclastogenesis and/or reduce osteoclast activation. The term “inhibit” as used herein can refer to the detectable reduction and/or elimination of a biological activity exhibited in the absence of the agent. The term “effective amount” can refer to that amount of the agent necessary to achieve the indicated effect. The term “treatment” as used herein can refer to reducing or alleviating symptoms in a subject, preventing symptoms from worsening or progressing, or prevention of a disorder, progression of which depends on osteoclastogenesis and/or osteoclast activation. Examples of such disorders include osteolytic disease and/or bone loss or destruction in subjects with renal cancer, lung cancer, thyroid cancer, prostate cancer, multiple myeloma, breast cancer, osteoporosis, such as post-menopausal osteoporosis, Paget's disease, rheumatoid arthritis or head and neck squamous cell carcinoma.

The amount of the agent, that is both a CGT inhibitor and a glucosidase inhibitor, which can be administered to the cell culture or the animal is preferably an amount that does not induce any toxic effects which outweigh the advantages which accompany its administration.

Actual dosage levels of active ingredients in the pharmaceutical compositions may vary so as to administer an amount of the active compound(s) that is effective to achieve the desired therapeutic response for a particular patient.

The selected dose level may depend on the activity of the agent, the route of administration, the severity of the condition being treated, and the condition and prior medical history of the patient being treated. However, it is within the skill of the art to start doses of the compound(s) at levels lower than required to achieve the desired therapeutic effect and to gradually increase the dosage until the desired effect is achieved. If desired, the effective daily dose may be divided into multiple doses for purposes of administration, for example, two to four doses per day. It will be understood, however, that the specific dose level for any particular patient can depend on a variety of factors, including the body weight, general health, diet, time and route of administration and combination with other therapeutic agents and the severity of the condition or disease being treated. The adult human daily dosage may range from between about one microgram to about one gram, or from between about 10 mg and 100 mg, of the agent per 10 kilogram body weight. Of course, the amount of the agent which should be administered to a cell or animal can depend upon numerous factors well understood by one of skill in the art, such as the molecular weight of the agent and the route of administration.

Pharmaceutical compositions that are useful in the methods of the invention may be administered systemically in oral solid formulations, ophthalmic, suppository, aerosol, topical or other similar formulations. For example, it may be in the physical form of a powder, tablet, capsule, lozenge, gel, solution, suspension, syrup, or the like. In addition to the active agent, such pharmaceutical compositions may contain pharmaceutically-acceptable carriers and other ingredients known to enhance and facilitate drug administration. Other possible formulations, such as nanoparticles, liposomes resealed erythrocytes, and immunologically based systems may also be used to administer the agent. Such pharmaceutical compositions may be administered by a number of routes. The term “parenteral” used herein includes subcutaneous, intravenous, intraarterial, intrathecal, and injection and infusion techniques, without limitation. By way of example, the pharmaceutical compositions may be administered orally, topically, parenterally, systemically, or by a pulmonary route.

These compositions may be administered in a single dose or in multiple doses which are administered at different times.

The present invention can be illustrated in more details by the following example, however, it should be understood that the present invention is not limited thereto.

### EXAMPLE

Multiple myeloma (MM) is an incurable malignant disorder characterised by clonal expansion of plasma cells (PC) and debilitating osteolytic bone disease. In some cases, MM can start as an asymptomatic, pre-malignant disorder, called monoclonal gammopathy of undetermined significance (MGUS) which over time may transform to MM. Genetic defects and crosstalk of the malignant PC with the microenvironment are responsible, probably in equal measure, for the biological behaviour of MM.

### Genetic Basis of MM and the Role of the Microenvironment

Over-expression of different oncogenes, especially of cyclins D1, 2 or 3, often as a result of chromosomal translocations involving the IgH enhancer element, can be the primary genetic events in MM. These can be found even in patients with MGUS (Hideshima et al., Nature Reviews in Cancer 2, 927-937, 2002; Hideshima et al., Blood 104, 607-618, 2004). Disease progression can be dependent on acquisition of additional genetic alterations, such as C-MYC deregulation and inactivation of tumor suppressor genes such P53 or RB-related genes (Hideshima et al., Nature Reviews in Cancer, supra; Hideshima et al., Blood, supra; Mitsiades et al., Proceedings of the National Academy of Science USA 101, 540-545 2004). Direct interaction of MM cells with bone marrow stromal cells and osteoblasts (OB) can be the trigger for the production of a number of cytokines that in an auto- or paracrine fashion can promote survival and growth of the tumor itself and increased osteoclast (OC) activity with the consequent bone disease.

IL-6, one of the most important MM-trophic factors secreted by stroma and OB, can promote MM cell growth and survival by activation of the Ras/Maf/MAPK and JAK/STAT3 pathways respectively (Hideshima et al., Nature Reviews in Cancer, supra; Hideshima et al., Blood, supra; Mitsiades et al., Proceedings of the National Academy of Science USA, supra). Constitutional activation of the transcription factor NFkB in MM cells (Bharti et al., Blood, 103, 3175-3184 2004; Bharti et al., Journal of Biological Chemistry 279, 6065-6076 2004) as a result, amongst other cytokines and growth factors, of increased secretion of TNFa and insulin growth factor (IGF-1) by stroma can be critical for resistance to apoptosis and growth of the tumour cells (Mitsiades et al., Oncogene 21, 5673-5683 2002). Disruption of NFkB activation by the proteasome inhibitor bortezomib and its significant clinical activity highlight the pivotal role of NFkB in the biology of the myeloma PC (Hideshima et al., Journal of Biological Chemistry 277, 16639-16647, 2002). Thus, better understanding of the molecular interactions of MM-microenvironment is important for the development of novel therapeutics.

### Pathogenesis of Bone Disease in MM

Bone homeostasis can be achieved by the continuous and co-ordinated activities of two types of cells, the bone resorbing osteoclasts (OC) and bone forming osteoblasts. Osteolytic bone destruction in MM, one of the most debilitating complications of the disease, can be caused by enhanced activation of OC and late in disease, suppression of OB activity. There is evidence that this process is largely dependent on an intimate and physical proximity of MM cells with OC and OB and is mediated by myeloma or stroma cell-derived soluble factors. There is also evidence suggesting that the close interaction of MM cells with stroma, OC and OB can be important for myeloma survival and growth at least at the early stages of the disease. Therefore, disruption of this cross-talk can provide the potential of reducing tumour burden and severity of bone disease.

### Mechanisms of OC Activation in MM

RANKL is a surface-bound and/or in soluble cytokine that can function as a major OC-activating factor (OAF) in bone homeostasis (Boyle et al., Nature 423, 337-342, Wada et al., Trends in Molecular Medicine 12, 17-25, 2006). Its increased secretion by stroma cells and OB as well as by myeloma cells themselves can be the prime mechanism of OC activation, increased bone resorption and eventually osteolysis and bone disease in MM (De Leenheer et al., Current Opinion in Pharmacology 4, 340-346, 2004; Terpos et al., International Journal of Hematology 78, 344-348). Increased RANKL can be detected not only in the tumor microenvironment but also in the serum of patients with MM and as previously shown, increased RANKL/OPG (osteoprotegerin, an inhibitory decoy receptor of RANKL) is predictive of poor survival (Terpos et al., Blood, 102, 1064-1069, 2003). T cells, stimulated by myeloma-derived IL-7 cells, are also an important source of RANKL in multiple myeloma (Colucci et al., Blood 104, 3722-3730, 2004; Giuliani et al., Blood 100, 4615-4621, 2002). In addition to RANKL, increased secretion of the chemokine macrophage inflammatory protein-1a (MIP-1a) (Abe et al., Blood 100, 2195-2202, 2002; Choi et al., Journal of Clinical Investigation 108, 1833-1841, 2001), of cytokines such as IL-3 (Lee et al., Blood 103, 2308-2315, 2004) and VEGF (Dankbar et al., Blood 95, 2630-2636, 2000; Nakagawa et al., FEBS Letters 473, 161-164, 2000) in the myeloma microenvironment contribute to increased osteoclastic activity.

### Mechanisms of OB Inhibition in MM

Suppressed OB function and reduced bone forming activity can be a compounding factor contributing to bone disease in late MM. Attention has been drawn to the increased levels of Dickkopf (Dick), a Wnt pathway soluble inhibitor, found increased in the peripheral blood and bone marrow plasma of patients with MM. The canonical Wnt pathway is required for OB development and function and its inhibition by Dick appears to be an important factor in OB dysfunction in MM. OB dysfunction is also imparted by other soluble factors secreted in excess in the MM microenvironment, such as IL-3 and HGF.

### Glycosphingolipids and Malignancy

Glycosphingolipids (GSL) are complex lipids, which constitute of the cellular plasma membrane generated from glycan modification of ceramide (Degroote et al., Seminars in Cell and Developmental Biology 15, 375-387, 2004).

Structurally GSLs can vary between tissues and also within the same tissue during differentiation. This variability can reflect their differing functional roles in many cellular processes including modification of cell signaling initiated by tyrosine kinases at the cell membrane, cell cycle control and apoptosis, adhesion and migration ((Degroote et al., supra). Quantitative and/or qualitative changes in the cellular GSL profile can be a trait of malignant transformation (Hakomori, Glycoconjugate Journal 17, 627-647, 2000).

In an large number of in vitro and in vivo pre-clinical studies, tumor-associated GSL have been shown to modulate a number of cellular functions that promote tumor, survival and growth, metastasis, angiogenesis and induce suppression of the anti-tumor immunity (Birkle et al., Biochimie 85, 455-463, 2003; Hakomori, Proceedings of the National Academy of Sciences USA 99, 10231-10233 2002). Thus, altered GSL composition can be not just a neutral process associated with malignant transformation; instead it participates and enhances cellular processes that are crucial for the clinical behaviour of a given tumor.

### Osteoclast Development Function and GSL

Lactosylceramide, GM2 and GM3 can be the main GSL constituents of mature osteoclasts, while GM1 can co-localize with RANK, the RANKL receptor, in lipid rafts Inhibition of GSL synthesis by the glycosylceramide synthase inhibitor d-PDMP or chemical disruption of lipid rafts can prevent OC development. Also pertinent to the pathogenesis of bone disease in MM, Iwamoto et al., have demonstrated an in vitro synergistic effect of exogenous lactosylceramide in RANKL-dependent osteoclastogenesis (Iwamoto et al., Journal of Biological Chemistry 276, 46031-46038, 2001).

### SUMMARY

Iminosugars that are ceramide glucosyltransferase (CGT) inhibitors may be of benefit in reducing OC activation by preventing generation of tumour-derived, pre-osteoclastogenic GSL as well as by inhibiting de novo OC GSL synthesis and thus OC activation. However, this mechanism does not fully explain the reduction in activation using NB-DNJ since PDMP, a known CGT inhibitor, was as effective but the more selective imino sugar inhibitor, NB-DGJ (Andersson et al., Biochemical Pharmacology 59, 821-829, 2000), was significantly less potent. Deoxynojirimycin (DNJ) analogues including NB-DNJ have both α- and β-glucosidase inhibitory activities in addition to their inhibitory effects on CGT (Platt et al., Journal of Biological Chemistry 269, 27108-27114, 1994). The reduction in osteoclast activation by DNJ iminosugars may suggest that more than one known mechanism, can play a role in the activation pathway.

A number of iminosugars are known CGT and glucosidase inhibitors (Butters et al., Chemical Reviews 100, 4683-4696, 2000) and one, N-butyl-deoxynojirimycin (NB-DNJ), has found clinical utility for reducing GSL biosynthesis to control the lysosomal accumulation of GSL in Gaucher disease. Such iminosugars may be useful for treating disorders, where osteoclast activation may be the primary effect in disease proliferation. One example of these disorders may be MM, where significant bone destruction is observed.

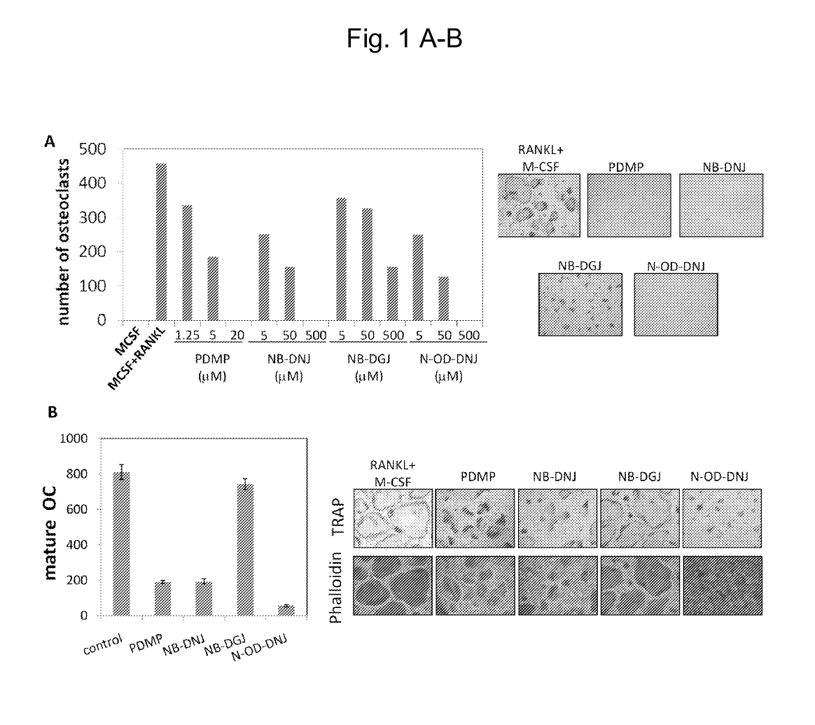

FIG. 1 presents data for in vitro inhibition by selected iminosugars of RANKL-dependent osteoclastogenesis.

A. Mouse bone marrow cells were cultured in the presence of 25 ng/ml M-CSF (macrophage colony stimulating factor) with 50 ng/ml RANKL and with or without d-PDMP (1.25, 5 or 20 μM), NB-DNJ (N-butyl-deoxynojirimycin), NB-DGJ (N-butyl-deoxygalactonojirimycin), or N-OD-DNJ (N-octadecyl-deoxynojirimycin) (5, 50 or 500 μM) in 96-well plates for 4 days. Cultures on plastic plates were fixed and stained for TRAP (tartrate resistant acid phosphate). TRAP positive multi-nuclear (>3 nuclei) osteoclast cells were counted.

B. Mouse bone marrow cells were cultured in the presence of 25 ng/ml M-CSF with 50 ng/ml RANKL in 48-well plates. On day 3 d-PDMP (1.25, 5 or 20 μM), NB-DNJ, NB-DGJ, or N-OD-DNJ (5, 50, or 500 μM) were added. Cells were cultured for another 24 hours before fixed and stained for TRAP. TRAP positive osteoclast cells mature were counted on day 4 (left). Morphology of 4 day osteoclasts. Cells were stained with either TRAP or phalloidin to demonstrate osteoclasts and F-actin respectively.

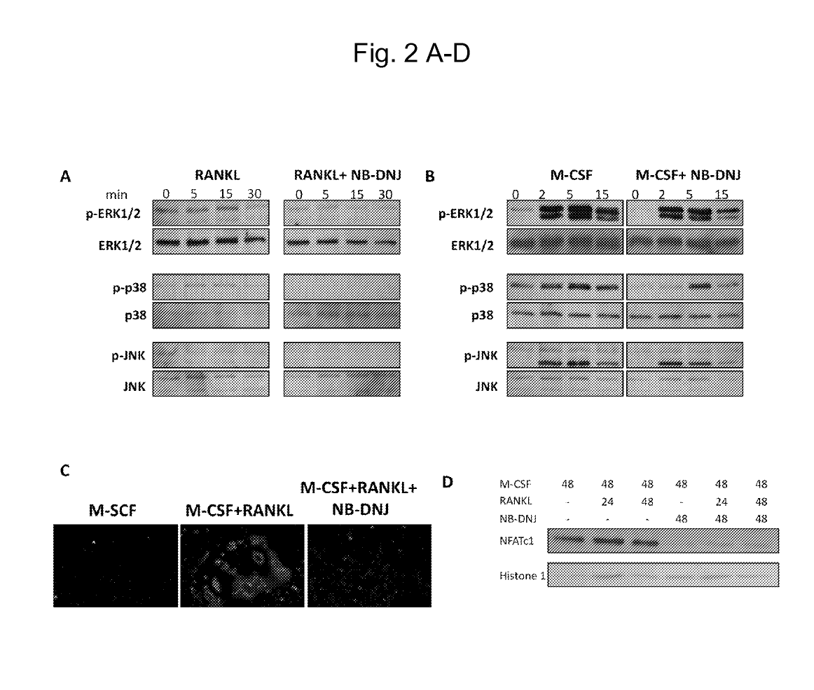

FIG. 2 presents data for inhibition of MAPK signalling and NFATc activation during osteoclastogenesis for selected iminosugars.

BMCs were cultured to day 3 then starved in 0.5% serum medium overnight. Cells were treated with RANKL (A) or M-CSF (B) for the time points indicated and then immunoblotted with α-pERK1/2, α-pP38, α-pJNK antibodies. Membranes were stripped and restained with α-ERK, α-P38, α-JNK antibodies. M-CSF and RANKL-dependent phosphotylation of p38 and to a lesser extend of ERKa,d Jnk is observed upon treatment with NB-DNJ C. Overnight serum starved OC were treated as indicated as subsequently stained with anti-NFATc1 and viewed by immunofluorescent microscopy. NB-DNJ abrogated the M-CSF+RANKL-induced accumulation of NFAtc1 in the nyclei. (D) BMCs were cultured 48 h with different combination of M-CSF, RANKL and NB-DNJ. Cells were collected and nuclear protein extracted and NFATc1 expression was checked by Western blot. Staining of histone-1 served as loading control. Considerably less nuclear NFTc1 is observed in the presence of NB-DNJ.

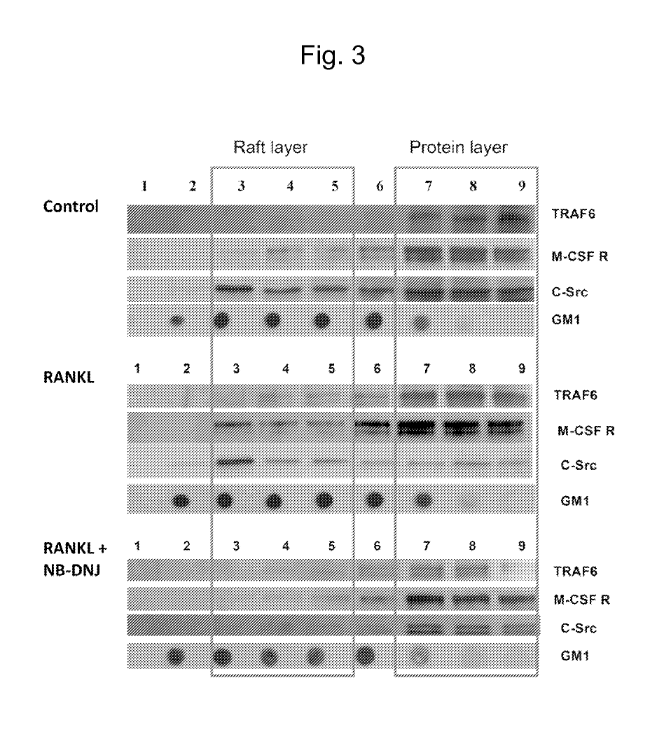

FIG. 3 presents data related to glycosphyngolipids perturbation of association of Src and TRAF6 with rafts.

Localization of TRAF6, Srcand GM1 gangliosides in lipid rafts (fraction 3-5) or non-raft fractions (fraction 7-9) in RAW 264.7 murine macrophage cells were cultured in medium only (control), in the presence of 50 ng/ml RANKL or RANKL+NB-DNJ in 48-well plates for 4 days. Cell lysates were prepared and separated using discontinuous sucrose gradient ultracentrifugation. A total of 9 fractions (1 ml each) were collected from the top to the bottom of the gradients, fraction 2 to 5 were positive for GM1 gangliosides, i.e. contained lipid rafts. The non-raft fractions were 7 to 9. Without RANKL, TRAF6 localises in the non-raft fraction while Src is presented in both raft and nonraft fractions. After RANKL treatment, TRAF6 was detected in raft fraction and Src almost totally shifted into the raft fractions. In the presence of NB-DNJ, TRAF6 and Src are excluded from the rafts and thus cannot interact with RANKL.

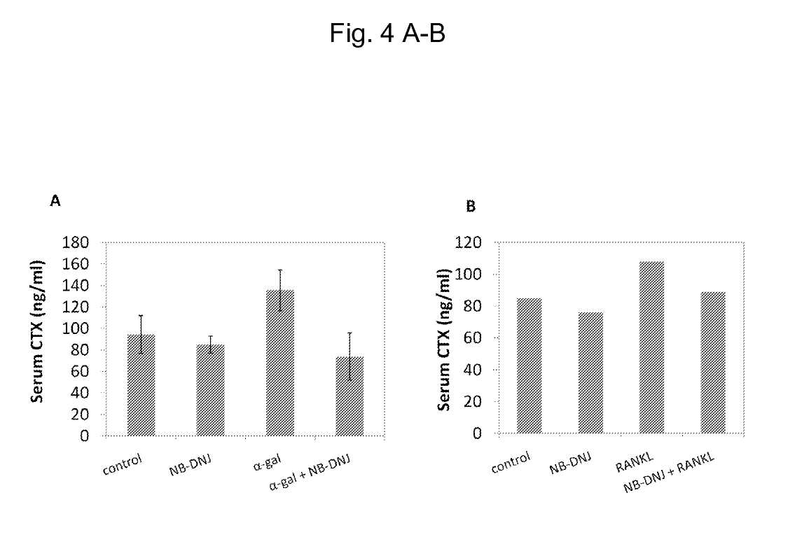

FIG. 4 A-B presents data for in vivo inhibition by selected iminosugars of osteoclast activation by galactosylceramide and RANKL.

A. NB-DNJ inhibits a-galactosylceramide-induced OC activation as reflected by serum CTX levels. NB-DNJ (500 mg/Kg) or PBS were injected intraperitoneal (i.p.) once a day for 6 consecutive days in 8 wk old C57BL/6 mice. Alpha-galactosylceramide or PBS was administered by a single i.p. injection of 2 μg on day 3. Serum collected on day 7 was used to determine carboxyterminal cross-linking telopeptide of type 1 collagen (CTX) levels (n=4-5 8 wk old female C57BL/6 mice).

B. NB-DNJ inhibits RANKL-induced OC activation as reflected by serum CTX levels The same schedule of NB-DNJ as above was used with RANKL at 4 mg/day administered i.p from day 3 (n=2).

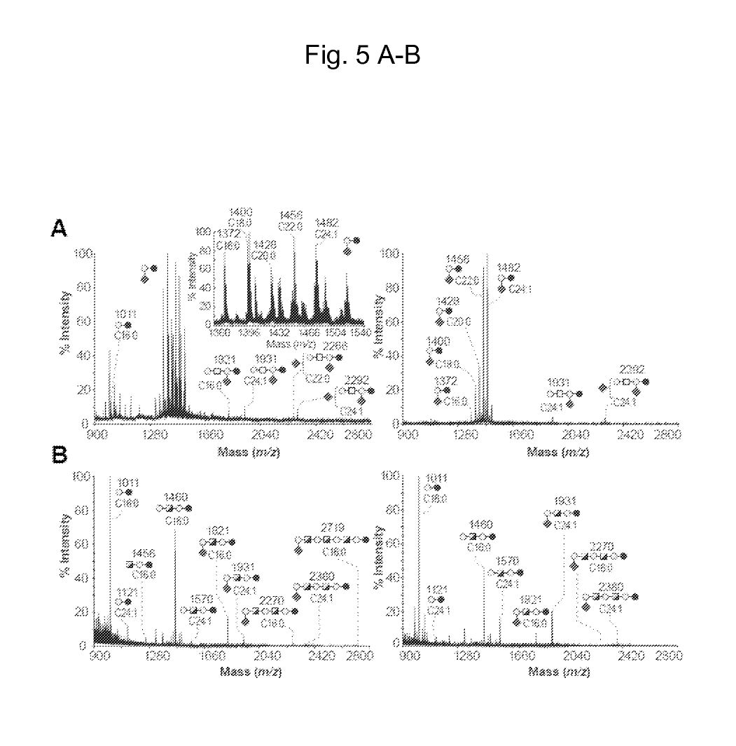

FIG. 5 A-B present mass spectral profiles of GSL in multiple myeloma (MM) patients. The profiles reveal that GM2 and GM3 are most prevalent GSL in MM.

Upper-phase GSLs from (A) MM patient CD138⁺ and (B) MM patient CD138⁻ bone marrow cells. Profiles of GSLs are from the 80% (left panels) and 100% propanol (right panels) fractions from a C₁₈ Sep-Pak. Inset corresponds to zoomed scan of the GM₃ cluster area.

GSLs are indicated as cartoon structures for the glycan moiety and composition of the fatty acid for the lipoform moiety, considering d-erythro-sphingosine as the sphingosine base. Cartoon structures are according to the Consortium for Functional Glycomics (http://www.functionalglycomics.org) guidelines. Fatty acid composition is indicated underneath the cartoon structure. Unassigned peaks correspond to chemical derivatization artefacts and/or to structures not corresponding to GSLs. All molecular ions are [M+Na]⁺. Structural assignments of the glycan moieties are based on monosaccharide composition, tandem mass spectrometry and knowledge of biosynthetic pathways.

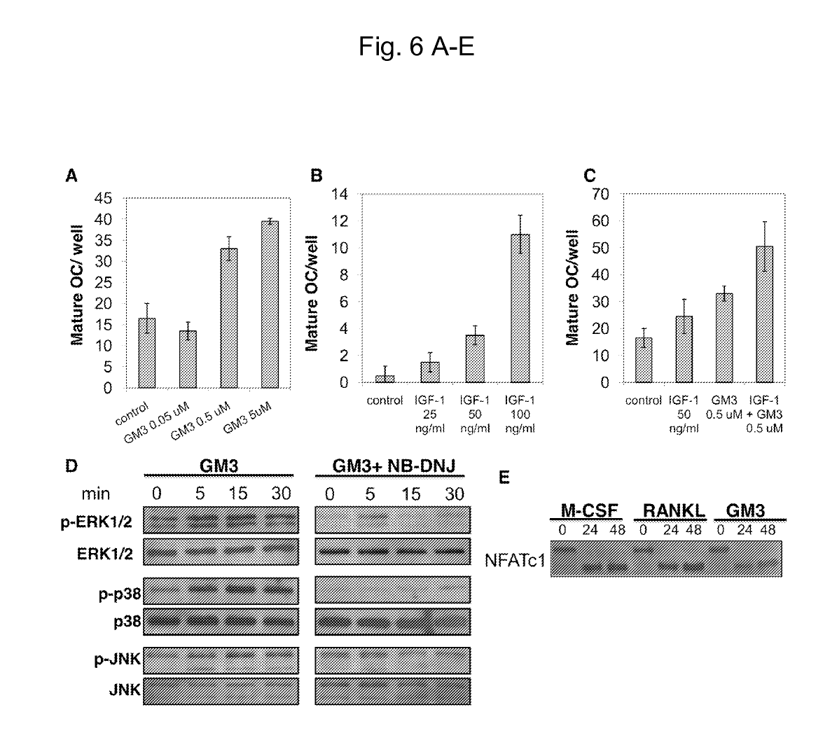

FIG. 6 A-E show data demonstrating that GM3 cooperates with RANKL and IGF-1 in promoting osteoclastogenesis.

A. Mouse bone marrow cells were cultured in the presence of 25 ng/ml M-CSF with 50 ng/ml RANKL and GM3 (0.05, 0.5 or 5 μM) in 48-well plates for 4 days. Cultures on plastic plates were fixed and stained for TRAP. TRAP positive mature osteoclast cells were counted.

B. IGF-1 promotes osteoclastogenesis. As well as RANKL+M-CSF, IGF-1 at the indicated concentrations was added.

C. IGF-1 co-operates with GM3 in promoting osteoclastogenesis. OC were developed in the presence of RANKL+M-CSF (control), or these two cytokines plus IGF-1, GM3 or IGF-1+GM3.

D. OC were cultured with M-CSF+RANKL to day 3 then starved in 0.5% serum medium overnight. Cells were treated with GM3 or GM3+NB-DNJ for the time points indicated and then immunoblotted with α-pERK1/2, α-pP38, α-pJNK antibodies. Membranes were stripped and restained with α-ERK, α-P38, α-JNK antibodies. GM3 promotes phosphorylation of EER, P38 and JNK an effect abrogated by NB-DNJ.

E. Overnight serum-starved OC were treated either with M-CSF, RANKL or GM3 and immunoblotting was performed against NFATc1 at the indicated time-points. GM3 is as effective as M-CSF and RANKL NFATc1 into its transcriptional in dephosphorylated (lower band) active form.

TABLE 1

| | % relative intensity** | | | | | | | | |
|---|---|---|---|---|---|---|---|---|---|
| | CD138+ | CD138− | CD138− | CD138− | | | | | |
| GSL* | SK0127 | RB5147 | SM0648 | GC3427 | KMS11 | KMS12 | RPMI8226 | U266 | JJ3N |
| Lactosyl | 2 | 98 | 100 | 100 | | 1 | 9 | | 20 |
| | 4 | 100 | 100 | 100 | | 3 | 2 | | 0 |
| GM3 | 100 | | 0 | 8 | 5 | 2 | 100 | 17 | 100 |
| | 100 | | 6 | 11 | 5 | 22 | 56 | 15 | 100 |
| GM2 | | | | | 100 | 100 | 60 | 100 | 90 |
| | | | | | 100 | 100 | 100 | 100 | 67 |

*Corresponds to the glycan moiety of the GSLs.
**% Relative intensity was calculated as follows: All spectra were subjected to peak deisotoping. The % relative intensity of all GSLs that corresponded to the same glycan moiety with all possible ceramide moieties (lipoforms) were summed up. The summed up % relative intensities of all GSLs in the same spectrum were normalized (100%) to the maximum relative intensity.

### Discussion

In Multiple myeloma (MM), a malignancy of plasma cells, autocrine and paracrine networks involving the malignant cells and their microenvironment, especially osteoclasts (OC), can play a crucial role in disease pathogenesis. OC activation and bone destruction are common and devastating events in this disease. Tumor-derived glycosphingolipids (GSL) have been shown to modify the tumor microenvironment by promoting tumor growth, angiogenesis, immune evasion and metastasis. The role of MM-derived GSL in OC development and activation was investigated and the role of de novo GSL synthesis in OC development was defined. Using MALDI-TOF MS and MS-MS (see Table 1 and FIG. 5), the GSL repertoire of primary CD138+ myeloma cells (n=3) was determined and compared it with non-myeloma bone marrow cells (i.e., without CD138+ cells) (n=3), and various myeloma cell lines (n=5). GM3 was found to be the dominant GSL in primary myeloma cells and GM2/GM3 in myeloma cell lines; by contrast, in non-myeloma marrow the non-polar LacCer was the dominant GSL. As GM3 was the dominant GSL in myeloma cells, the effect on osteoclast function was tested (FIG. 6). Exogenous GM3 was found to synergistically enhance the ability of M-CSF and RANKL to induce maturation of murine bone marrow OC in vitro. This, as shown by immunoblotting, was associated with increased ERK1/2, p38, JNK phosphorylation and NFATc dephosphorylation, signal transduction and transcriptional events respectively, required for OC differentiation and maturation in response to RANKL. Furthermore, GM3 further enhanced OC maturation in synergy with IGF-1, a growth factor known to promote myeloma growth and OC activation (see FIG. 6). Next, the effect of inhibition of de novo GSL biosynthesis on osteoclastogenesis was tested. The glucose ceramide synthase inhibitor (CGT) NB-DNJ was found to inhibit RANKL- and M-CSF-dependent development of murine as well as human, monocyte-derived OC in a dose dependent manner, when added either in the beginning or during OC differentiation cultures (FIG. 1). This effect was associated with significantly reduced RANKL- and M-CSF-dependent phosphorylation of ERK, JNK and p38 as well as reduced localisation of NFATc in the nucleus (FIG. 2). OC development in response to RANKL-RANK interaction requires movement of RANK into lipid rafts where it interacts with TRAF6, an adaptor crucial for downstream signalling and with cSrc which is required for actin ring formation and OC resorptive activity. Using sucrose gradient membrane fractionation and GM1 as a marker of rafts, it was found that GCS inhibitors partially disrupt the integrity of lipid rafts in developing osteoclasts and prevent RANKL-induced localisation of TRAF6 and Src in lipid rafts (FIG. 3). Analysis of the ability of NB-DNJ to block OC activation caused by a single injection of alpha-galactosylceramide (αGC), an invariant NKT cell ligand known to rapidly activate the innate immune response was performed in vivo (Fg 4). It was found that in mice receiving αGC, serum C-telopeptide Type I collagen (CTX) levels increased by ˜50% (p<0.01), while in mice co-injected with αGC plus NB-DNJ (daily for 3 days i.p) CTX levels returned to baseline (p<0.01); in mice receiving only NB-DNJ, CTX levels were not significantly different to vehicle controls (p>0.05) (see FIG. 4). Taken together, these data demonstrate a novel role of GSL in promoting OC differentiation and activation. Thus, certain iminosugar inhibitors may be of benefit in reducing OC activation and bone destruction in MM by preventing generation of tumor-derived, pre-osteoclastogenic GSL as well as by inhibiting de novo OC GSL synthesis and thus OC activation.

Although the foregoing refers to particular preferred embodiments, it will be understood that the present invention is not so limited. It will occur to those of ordinary skill in the art that various modifications may be made to the disclosed embodiments and that such modifications are intended to be within the scope of the present invention.

All of the publications, patent applications and patents cited in this specification are incorporated herein by reference in their entirety.

What is claimed is:

1. A method for inhibiting osteoclastogenesis and/or reducing osteoclast activation, comprising:
administering to a subject in need thereof an effective amount of an agent, which is a ceramide glucosyltransferase inhibitor and a glucosidase inhibitor.

2. The method of claim 1, wherein the agent is an iminosugar.

3. The method of claim 1, wherein the agent is a compound of formula I: 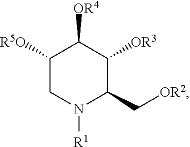
a pharmaceutically acceptable salt or a prodrug thereof, wherein R¹ is selected from alkyls, cycloalkyls, aryls, alkenyls, acyls, aralkyls, aroyl, alkoxy groups, aralkoxy groups and heterocyclic groups and wherein R², R³, R⁴, and R⁵ are each independently selected from hydrogen, acyl groups, alkanoyl groups, aroyl groups, and haloalkanoyl groups.

4. The method of claim 3, wherein R², R³, R⁴, and R⁵ are each hydrogen.

5. The method of claim 4, wherein R¹ is C₁-C₂₄ alkyl group.

6. The method of claim 5, wherein R¹ is C₂-C₁₂ alkyl group.

7. The method of claim 6, wherein R¹ is C₃-C₅ alkyl group

8. The method of claim 7, wherein R¹ is butyl.

9. The method of claim 5, wherein R¹ is C₁₄-C₂₂ alkyl group.

10. The method of claim 9, wherein R¹ is C₁₇-C₂₀ alkyl group.

11. The method of claim 10, wherein R¹ is octadecyl.

12. The method of claim 1, wherein the subject is a human being.

13. The method of claim 1, wherein said subject has a condition selected from multiple myeloma, renal cancer, lung cancer, thyroid cancer, prostate cancer, breast cancer, osteoporosis, Paget's disease, rheumatoid arthritis and head and neck squamous cell carcinoma and said administering reduces in the subject bone destruction associated with said condition.

14. The method of claim 13, wherein the subject has multiple myeloma.

15. A method of reducing or preventing osteolytic activity and/or bone loss, comprising:
administering to a subject in need thereof an effective amount of an agent, which is a ceramide glucosyltransferase inhibitor and a glucosidase inhibitor.

16. The method of claim 15, wherein the agent is an iminosugar.

17. The method of claim 15, wherein the agent is a compound of formula I: 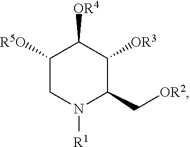
a pharmaceutically acceptable salt or a prodrug thereof, wherein R¹ is selected from alkyls, cycloalkyls, aryls, alkenyls, acyls, aralkyls, aroyl, alkoxy groups, aralkoxy groups and heterocyclic groups and wherein R², R³, R⁴, and R⁵ are each independently selected from hydrogen, acyl groups, alkanoyl groups, aroyl groups, and haloalkanoyl groups.

18. The method of claim 17, wherein R², R³, R⁴, and R⁵ are each hydrogen.

19. The method of claim 18, wherein R¹ is C₁-C₂₄ alkyl group.

20. The method of claim 19, wherein R¹ is C₂-C₁₂ alkyl group.

21. The method of claim 20, wherein R¹ is C₃-C₅ alkyl group

22. The method of claim 21, wherein R¹ is butyl.

23. The method of claim 19, wherein R¹ is C₁₄-C₂₂ alkyl group.

24. The method of claim 23, wherein R¹ is C₁₇-C₂₀ alkyl group.

25. The method of claim 24, wherein R¹ is octadecyl.

26. The method of claim 15, wherein the subject is a human being.

27. The method of claim 15, wherein said subject has a condition selected from multiple myeloma, renal cancer, lung cancer, thyroid cancer, prostate cancer, breast cancer, osteoporosis, Paget's disease, rheumatoid arthritis and head and neck squamous cell carcinoma and said osteolytic activity and bone loss are caused by said condition.

28. The method of claim 27, wherein the subject has multiple myeloma.
